(19) United States
(12) Patent Application Publication
(10) Pub. No.: US 2009/0074842 A1
(43) Pub. Date: Mar. 19, 2009

(54) Medicament patch

(76) Inventor: Connie Hsu, Tainan City, CA (US)

Correspondence Address: Chiayuan Chang, 1217 Beach Park Blvd., Foster City, CA, 94404, US

(21) Appl. No.: 12/222,181

(22) Filed: Aug. 4, 2008

(30) Foreign Application Priority Data
Sep. 13, 2007 (TW) 096215415

(51) Int. Cl. A61K 9/70; A61P 21/00; A61P 19/00

(52) U.S. Cl. 424/443

(57) ABSTRACT

A medicament patch comprises a release paper that oppositely lies on the surface of a patch carrier. The release paper is defined to, from right to left, a first teardown slice, a second teardown slice, and a third teardown slice. Each teardown-slice correspondingly matches with an adjacent edge of another teardown slice through joints of concaves and convexes. A radian that is inwards depressed by degree is formed where the second teardown slice is close to the adjacent edges of the first teardown slice and third teardown slice. A curve is then formed to make the release paper stably and securely adhere to a carrier. However, for the functional effect of a product, the curve is unlikely complicated to make a trouble to a user in tearing down a medicament patch.

### BACKGROUND OF THE INVENTION

1. Field of the Invention

This invention relates to a medicament patch and particularly to an improved medicament patch on which scrip may be easily tore down.

2. Description of the Related Art

Treating bone and muscle ache (Myalgia) or injury, since time immemorial, in Martial Arts Association or in a manner of so-called folk therapy, grass ointment is generally prepared, for example, containing gauleheria, menthol, eucalyptus oil, camphor and the like in order to relax and activate the tendons, and then a patch is used as a carrier for liniment. In consideration of citizen's welfare, such a medicament is brought into a category of medicine control. The patch containing the medicament treats and eases the body's ache, in which the medicament penetrates from a high-density portion of the body into a low-density portion of the body, and is then absorbed by the skin.

In order for the patch to be pasted, the medicament must be adhesive. Before being used, for fear of a substance of viscosity that is easily stained with other objects, the medicament patch is generally provided with a release paper to temporarily protect the medicament stained with the substance of viscosity; when the patch is used, the release paper is tore down and removed. For fear of the whole release paper that is large and thus not easily tore down, a medicament patch is designed, as shown in FIG. 6 and publicly disclosed in Taiwan Patent No. 552061. The medicament patch is a patent applied for a new style. However, on the medicament patch (1), a release paper (11) is arranged oppositely on a surface of the patch carrier (not shown because lying under the release paper). The release paper (11) is defined to, from right to left, a first teardown slice (111), a second teardown slice (112), and a third teardown slice (113). Each teardown-slice correspondingly matches with an adjacent edge of another teardown slice through joints of concaves and convexes. The second teardown slice (112) being close to the adjacent edges of the first teardown slice (111) third teardown slice (113), a plurality of convex unit margins (112A) that are opposite to convex unit margins (112B), and concave unit margins (112C) that are opposite to concave unit margins (112D) are respectively formed. However, although the variation of concaves and convexes, where the plurality of convex unit margins (112A) opposite to the convex unit margins (112B), and concave unit margins (112C) opposite to the concave unit margins (112D) may enhance the stability of adherence of the margins, a user might not tear it down smoothly.

Consequently, because of the technical defects of described above, the applicant keeps on carving unflaggingly through wholehearted experience and research to develop the present invention, which can effectively improve the defects described above.

### SUMMARY OF THE INVENTION

In a conventional art, it is difficult to tear down a complicated release paper. In this invention, a curve is formed to make the release paper stably and securely adhere to a carrier. However, for the functional effect of a product, the curve is unlikely complicated to make a trouble to a user in tearing down a medicament patch.

In order to achieve the object mentioned above, the medicament patch according to this invention comprises a release paper that oppositely lies on the surface of a patch carrier. The release paper is defined to, from right to left, a first teardown slice, a second teardown slice, and a third teardown slice. Each teardown-slice correspondingly matches with an adjacent edge of another teardown slice through joints of concaves and convexes. In this invention, it is characterized that a radian that is inwards depressed by degree is formed where the second teardown slice is close to the adjacent edges of the first teardown slice and third teardown slice. Through experiment, the margin may be used to form a adhesive property and disengagement hysteresis, and thus it is ensured that the release paper does not easily shed off and easily tore down.

### BRIEF DESCRIPTION OF THE DRAWINGS

FIG. 1 is a 3D view of this invention;

FIG. 2 is a 3D view of a slightly cave and folded medicament patch according to this invention;

FIG. 3 is a 3D view of a second teardown slice according to this invention that is tore down;

FIG. 4 is a 3D view of the medicament patch in FIG. 3 that is turned, illustrating that an adhesive surface of the medicament patch is downwards;

FIG. 5 is a 3D view illustrating a first teardown slice that is tore down and attached for a next step of removal of a third slice; and

FIG. 6 is a plane view illustrating a prior art.

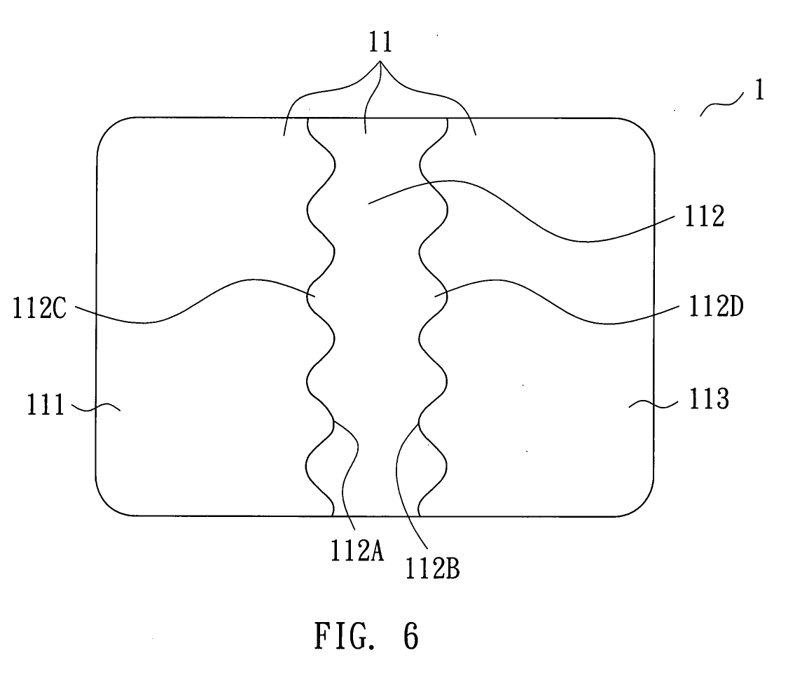

### DETAILED DESCRIPTION OF THE PREFERRED EMBODIMENTS

Now, the present invention will be described more specifically with reference to the following embodiments. It is to be noted that the following descriptions of preferred embodiments of this invention are presented herein for purpose of illustration and description only; it is not intended to be exhaustive or to be limited to the precise form disclosed.

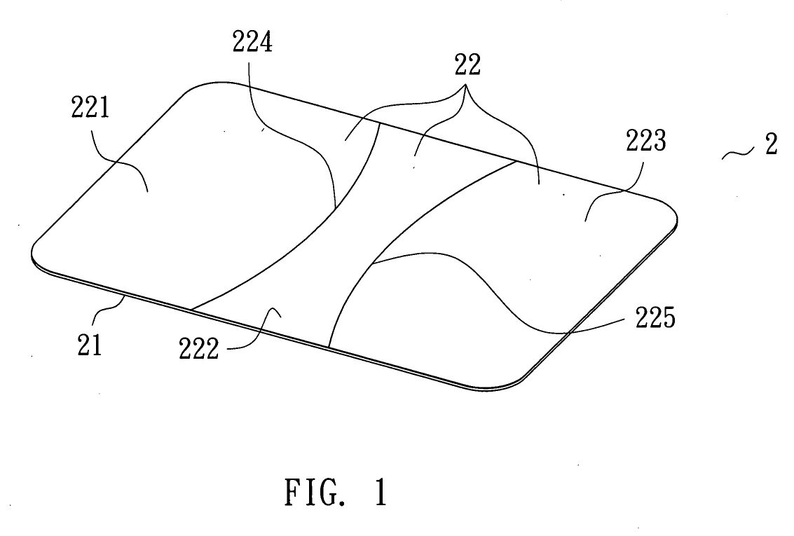

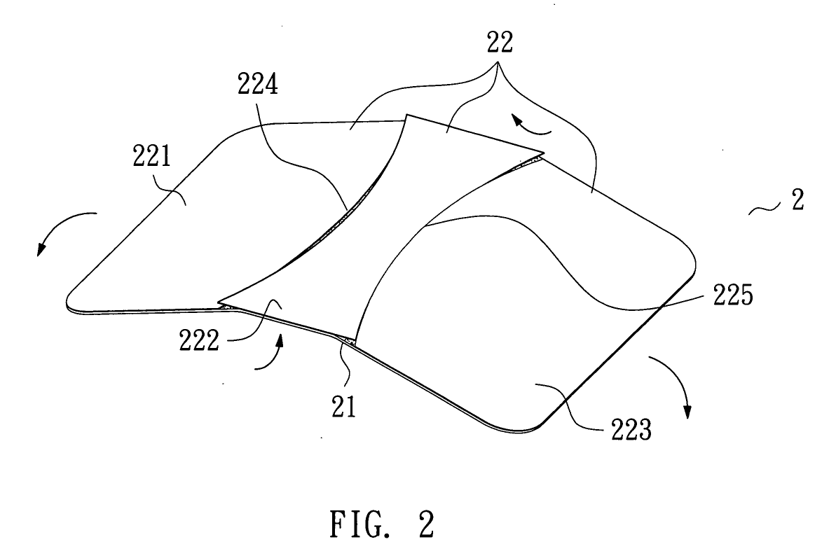

With reference to FIG. 1 and with cross-reference to FIG. 2, a medicament patch (2) according to this invention comprises a patch carrier (21) and a release paper (22).

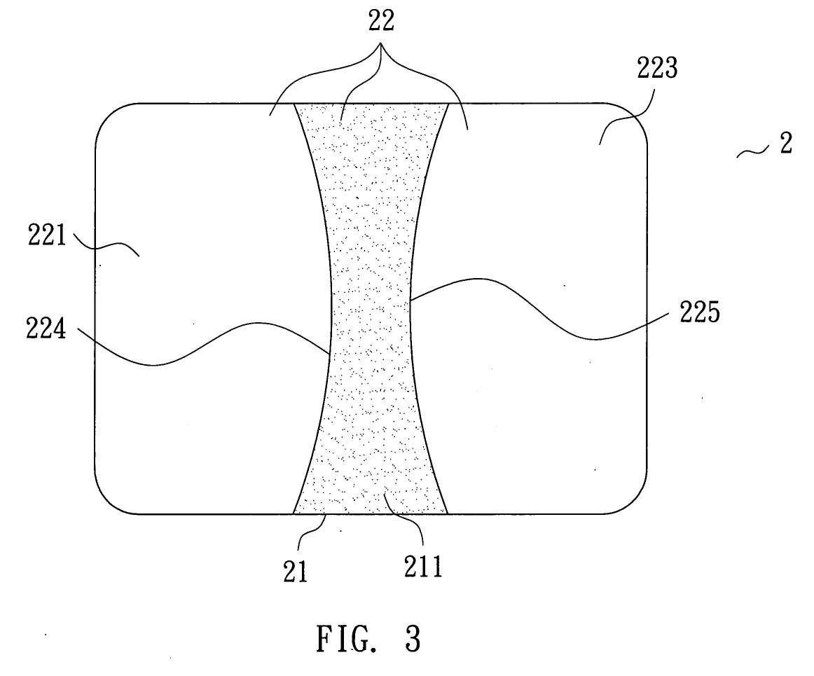

In FIGS. 2 and 3, an adhesive surface (211) of the patch carrier (21) is apparent. The patch carrier (21) contains the medicament that may penetrate into the body and is then absorbed by the skin to treat and ease the body's ache. In order for the patch to be pasted, the medicament must be adhesive.

The release paper (22) is on the basis of the viscosity of patch carrier (21). Before the patch carrier (21) is used, for fear of a substance of viscosity that is easily stained with other objects, the release paper (22) oppositely lies on the surface of a patch carrier (21). The release paper (22) is defined to, from right to left, as shown in FIG. 2 for a basis, a first teardown slice (221), a second teardown slice (222), and a third teardown slice (223). Each teardown-slice correspondingly matches with an adjacent edge of another teardown slice through joints of concaves and convexes. A radian that is inwards depressed by degree is formed where the second teardown slice (222) is close to the adjacent edges (224) of the first teardown slice (221) and third teardown slice (223).

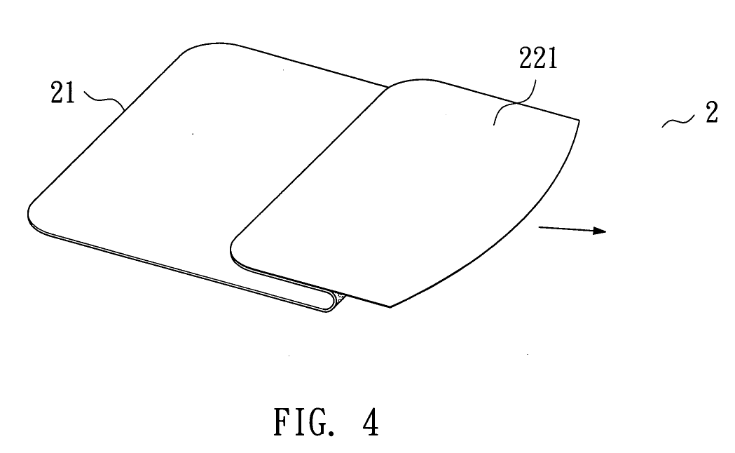

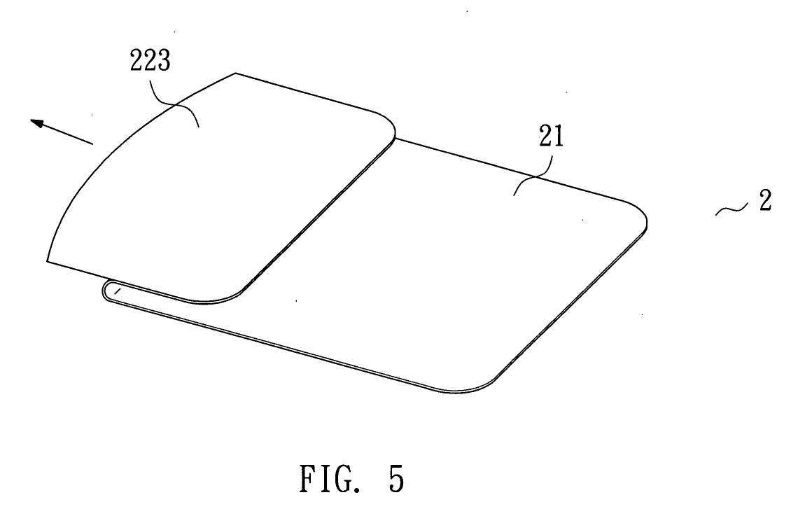

Refer to FIGS. 3 through 5 illustrating serial steps of operation of this invention. FIG. 3 illustrates the second teardown slice (222) according to this invention that is tore down. FIG. 4 illustrates that after the patch according to this invention shown in FIG. 3 is turned, the medicament patch (2) turns the adhesive surface (211) to a downward portion, the skin of human body, for a next step of removal of the first teardown slice (221). FIG. 5 illustrates the first teardown slice (221) that is tore down and attached for a next step of removal of the third slice (223).

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A medicament patch, comprising a release paper that oppositely lies on the surface of a patch carrier and is defined to, from right to left, a first teardown slice, a second teardown slice, and a third teardown slice, each of which correspondingly matches with an adjacent edge of another teardown slice through joints of concaves and convexes, and being characterized that:
a radian that is inwards depressed by degree is formed where the second teardown slice is close to the adjacent edges of the first teardown slice and third teardown slice.
